(19) United States
(12) Patent Application Publication
(10) Pub. No.: US 2015/0219615 A1
(43) Pub. Date: Aug. 6, 2015

(54) QUANTITATION OF NAPHTHENIC ACIDS IN WATER AND CRUDE OIL

(71) Applicant: PHILLIPS 66 COMPANY, Houston, TX (US)

(72) Inventors: Leonard NYADONG, Owassa, OK (US); Herbert R. PINNICK, JR., Bartlesville, OK (US); Carolyn JOHLMAN, Bartlesville, OK (US)

(73) Assignee: PHILLIPS 66 COMPANY, Houston, TX (US)

(21) Appl. No.: 14/609,932

(22) Filed: Jan. 30, 2015

Related U.S. Application Data
(60) Provisional application No. 61/935,994, filed on Feb. 5, 2014.

(51) Int. Cl. G01N 33/28; G01N 30/72; G01N 33/18

(52) U.S. Cl. CPC G01N 33/2835; G01N 33/1826; G01N 30/7266

(57) ABSTRACT

The present disclosure relates generally to processes for accurate quantitation of naphthenic acids in liquid samples. The method allows efficient identification as well as quantitation of NA species based on carbon number and ring structure, but requires no chemical modification or extraction of the sample allowing a rapid throughput. Reverse phase liquid chromatographic separation of the sample minimizes (or eliminates) matrix suppression effects to allow detection of all NAs present in various samples by mass spectroscopy with superior detection thresholds that are as much as 350-fold lower than conventional methods.

### CROSS-REFERENCE TO RELATED APPLICATIONS

This application is a non-provisional application which claims benefit under 35 USC §119(e) to U.S. Provisional Application Ser. No. 61/935,994 filed Feb. 5, 2014, entitled QUANTITATION OF NAPHTHENIC ACIDS IN WATER AND CRUDE OIL, which is incorporated herein in its entirety.

### STATEMENT REGARDING FEDERALLY SPONSORED RESEARCH OR DEVELOPMENT

None.

### FIELD OF THE INVENTION

The present invention relates to method for quantitation of naphthenic acids in process water and crude oil extracts.

### BACKGROUND

Naphthenic acids (NAs) are found to varying degrees in crude oil, and are predominantly responsible for corrosion and fouling of process equipment in refineries, oil production operations and pipelines. The high aqueous solubility of NAs makes them one of the major dissolved organic constituents in refinery process waters, and due to their biologic toxicity, must be removed from refinery wastewater prior to release into the environment. Therefore, rapid characterization and quantitation of NAs in both petroleum fractions as well as refinery process water is essential.

NAs are typically quantitated by gas chromatography-mass spectrometry (GC/MS) and by two-dimensional gas chromatography (GC-GC) followed by MS. Both methods require a complex and lengthy sample preparation procedure which includes extraction of the NAs into a suitable solvent, followed by derivatization prior to analysis. The gas chromatography analysis usually requires a lengthy temperature program depending on the complexity of the sample for the elution of all components from the column.

Thus, there is a need for processes that can more rapidly identify and quantitate the levels of specific NAs in a sample. Such a method would both decrease cost and make practical more frequent analysis of water and crude petroleum samples. One benefit of more frequent testing is greater assurance that process water contaminated with naphthenic acids is not released to the environment. Monitoring the levels of naphthenic acids in process water also allows development of strategies for process water remediation and/or recycling, as well as mitigation strategies to prevent fouling of refinery process equipment.

### BRIEF SUMMARY OF THE DISCLOSURE

The present disclosure presents unique processes for characterizing and quantifying NAs present in refinery waste waters and crude oil fractions. The inventive process allows accurate quantitation of one or more naphthenic acids in a liquid sample. Certain embodiments comprise: subjecting a portion of the liquid sample to reverse phase high performance liquid chromatography utilizing a chromatography column capable of separating molecules having a molecular weight of 2,000 or less to produce a chromatography effluent comprising at least partially separated naphthenic acids, each naphthenic comprising from 2 to 25 carbons; directing the chromatography effluent to an electrospray ionization source to produce negatively charged naphthenic acid ions that are analyzed on a mass spectrometer to produce a raw mass spectrum; correcting the raw mass spectrum by multiplying the intensity of one or more spectral peaks corresponding to the one or more naphthenic acids by a response factor, where the response factor is empirically-determined via measurement of the relative mass spectrum peak intensity of known quantities of carboxylic acid standards comprising between 2 and 25 carbons, the response factor being dependent upon the number of carbons in the carboxylic acid standards, the number of ring structures in the carboxylic acid standards, or both. The response factor corrects the signal magnitude of the one or more spectral peaks corresponding to the one or more naphthenic acids, thereby allowing more accurate quantitation of the one or more naphthenic acids in the liquid sample.

In certain embodiments, the process additionally comprises correcting the raw mass spectrum by subtracting a blank mass spectrum, or the average of two or more blank mass spectrums, thereby allowing more accurate quantitation of the one or more naphthenic acids. In certain embodiments, the chromatography effluent is eluted from the column utilizing an increasing organic solvent gradient. In certain embodiments, the organic solvent is methanol.

### BRIEF DESCRIPTION OF THE DRAWINGS

A more complete understanding of the present invention and benefits thereof may be acquired by referring to the follow description taken in conjunction with the accompanying drawings in which:

FIG. 1 depicts a representative total ion chromatogram trace (negative electrospray ionization) of a sample produced by gradient elution HPLC.

FIG. 2 demonstrates a more detailed mass spectra analysis of the total ion chromatogram trace shown in FIG. 1.

FIG. 3 shows selected gradient elution HPLC ESI chromatogram traces corresponding to acyclic naphthenic acids possessing 6, 9 and 11 carbons, respectively.

FIG. 4 shows the selected ESI chromatogram traces for three detected naphthenic acid species having the same carbon number (n=12) but different z numbers (−8, −4, and 0) corresponding to NAs structures with 4-rings, 2-rings and acyclic, respectively.

FIG. 5 is a stacked bar graph that depicts the distribution of naphthenic acids (by total carbon number) in a refinery-produced water sample.

The invention is susceptible to various modifications and alternative forms, specific embodiments thereof are shown by way of example in the drawings. The drawings may not be to scale. It should be understood that the drawings and their accompanying detailed descriptions are not intended to limit the scope of the invention to the particular form disclosed, but rather, the intention is to cover all modifications, equivalents and alternatives falling within the spirit and scope of the present invention as defined by the appended claims.

### DETAILED DESCRIPTION

The methods described herein are based on gradient elution high performance liquid chromatography-mass spectrometry (HPLC MS) in which samples are spiked with an isotope labeled internal standard and then injected onto a reversed-phase HPLC column. The method allows efficient identification as well as quantitation of NA species based on carbon number and ring structure. The method requires no chemical modification or extraction of the sample allowing a rapid throughput. The HPLC separation minimizes (or eliminates) matrix suppression effects to allow detection of all NAs present in various samples with superior detection thresholds that are as much as 350-fold lower than conventional methods, and detection specificity comparable to conventional direct sample injection methods.

Any HPLC column capable of separating small molecules of less than 2,000 mw may be utilized. Preferably, the HPLC column is a reverse-phase C-18 column, in which a hydrocarbon having an eighteen carbon backbone is typically bound to a silica or polymer substrate and preferentially attracts hydrophobic compounds, thereby retards their migration through the column. Hydrophobic compounds stick to reverse phase HPLC columns in solvents that are predominantly aqueous (i.e., polar), and are eluted from such columns with solvents that are predominantly non-polar. In RP HPLC compounds are separated based on their hydrophobic character.

Effective separation of molecules may be achieved by running a linearly-increasing gradient of the organic solvent mixed with a polar solvent (typically water) through the column over time. The result is a separation of individual NAs on the column such that they elute from the column at different times based on their carbon number, number of naphthenic rings and extent of alkyl branching. This allows more accurate quantitation of each NA present in the sample, as they can be quantitated with decreased signal interference on the mass spectrometer. The organic solvent may be any HPLC-grade organic solvent sufficient to elute NAs from the column. Typically the organic solvent is acetonitrile or methanol, but these examples do not serve to limit the scope of the invention. In certain embodiments, a small amount of an acid may be added to help distinguish peaks and may serve as a source of protons when the effluent is analyzed by electrospray ionization (ESI) mass spectrometry. Examples of acids that may be used include triflouroacetic acid, acetic acid and formic acid, although other acids may be effective as well.

Eluted NA are next directed to an ESI source. ESI technology is conventional in nature and thus will not be discussed in great detail herein. Basically, the chromatography effluent is nebulized in the ESI source (in negative ion mode) into highly negative-charged droplets followed by evaporation of ionized molecules from those droplets. A mass spectrometer analyzer subsequently detects the mass-to-charge (m/z) ratio of ionized molecules in the effluent, where m corresponds to mass and z corresponds to the charge.

In certain embodiments of the invention, quantitative analysis of NAs in a sample involves three steps. The data spectrum acquired by the mass spectrometer is first corrected by subtracting an average of the background spectrum values for triplicate injections of an H₂O blank into the system. The average mass spectrum across the chromatogram trace for the sample is then exported as peak intensity versus exact masses by use of commercial software supplied by the manufacturer of the mass spectrometer (X-calibur software, Thermo-Fisher Scientific). The mass spectrum is next corrected to normalize the signal response for the various mass spectral peaks based upon empirically-derived correlations between the magnitude of the spectrometer signal m, and the known m/z ratio for each NA species, where NAs are defined by the general formula CₙH_{2n+Z}O₂. The variable n indicates the carbon number and z is zero or a negative integer that specifies the hydrogen deficiency resulting from ring formation. In general, z number decreases by 2 for each additional ring structure in the NA. Finally, the use of a known quantity of an isotope-labeled (¹³C) internal standard allows quantitation of all NAs of interest in the sample.

To discuss the second step of the data correction process in greater detail, the spectral peak values empirically known to correspond to various NAs (according to the general formula CₙH_{2n-Z}O₂) are exported to a software spreadsheet program (e.g., Excel® by Microsoft, Inc.). Macros programmed into the spreadsheet program introduce a signal magnitude correction factor for low molecular weight NAs as well as a correction scheme for the signal magnitude of the internal standard. Typical corrected results are shown in Table 2. The quantity of each NA can then be calculated from the ratio of its associated mass spectral peak magnitude to that of a known amount of an isotope-labeled internal standard spiked into the sample. To accomplish this correction, the macro corrects the magnitude of each mass spectra peak corresponding to a NA of interest by multiplying the magnitude of each peak with a response factor (RF) according to the following expression:

Iₘ_{ᵢ}^{Cl}=Iₘ_{ᵢ}^{Avg}*RF

Where I^{Avg} is the average peak magnitude of a number of replicates at the i^{th} m/z (mass) and I^{C1} is the corrected peak magnitude at the i^{th} m/z. The response factors are empirically determined via analysis of a equimolar mixture of C6-C24 fatty acids.

As mentioned above, to accurately quantitate the concentration of various NA detected within a sample, the spectrum data are first corrected for the magnitude of a known quantity of isotope-labeled internal standard (IS). In certain embodiments, myristic acid-1-¹³C is utilized (IS+1 peak; m/z=229), although other radiolabeled NA or fatty acids can be utilized depending upon the molecular weight range of the sample to be analyzed. In embodiments where myristic acid-1-¹³C is utilized, the IS peak (m/z 228) is corrected for the contribution of the ¹³C peak magnitude of the IS-1 species (m/z 227) as follows:

I₂₈₈^{C2}=I₂₂₈^{C1}−x*I₂₂₇^{C1}x=0 . . . 100%

Where x is the calculated magnitude of the ¹³C peak of IS-1 (m/z=227) species. The magnitude of the IS+1 peak (m/z 229) is also corrected a second time for the contribution of the ¹³C peak magnitude of the IS species as follows:

I₂₈₉^{C2}=I₂₂₉^{C1}−y*I₂₂₈^{C2}y=0 . . . 100%

Where y is the calculated magnitude of the ¹³C peak of the IS (m/z=228) species. Hence, the concentration at the i^{th} mass (species), Cₘᵢ is computed as follows:

Cₘ_{ᵢ}=(Iₘ_{ᵢ}^{C1}/I₂₂₈^{C2})*C_{IS}

Table 2 shows an example of mass spectrometry data for one test sample, where the data has been corrected in the manner described above. The corrected data allows calculation of the concentration of various NA species that may be acyclic, or possess one or more ring structures (z=0 to −12). The first section (labeled “Acyclic”) represents the relative abundance of acyclic NA having from 4 to 25 carbons (left column), while the second section (labeled “One Double Bond Equivalent”) represents the relative abundance of NAs having a single ring structure (z=−2). The total weight percent of NA in each class (i.e., acyclic or one-ring) is displayed at the bottom of each spreadsheet section [Acyclic, One double bond equivalent, (DBE), etc.]. Similar calculations are made for larger multi-ring NAs having lower z numbers (e.g., −4, −6, etc.), but are not replicated here to prevent redundancy.

TABLE 2
Correcting mass spectroscopy data.

| | Acyclic | | | | | | One Double Bond Equivalent (DBE) | | | | | |
|---|---|---|---|---|---|---|---|---|---|---|---|---|
| | CₙH₂ₙO₂ | | | | | | CₙH₂ₙ₋₂O₂ | | | | | |
| | | Mass | Intensity | Corrected | | | | Mass | Intensity | Corrected | | |
| C | m/z | (avg) | (avg) | Intensity | Wt Pct | Conc. | m/z | (avg) | (avg) | Intensity | Wt PCt | Conc |
| 7 | 129.09 | 129.25 | 4.21E+02 | 1.35E+03 | 1.85 | 53.15 | 127.08 | 127.27 | 7.22E+01 | 2.31E+02 | 0.32 | 9.10 |
| 8 | 143.11 | 143.24 | 6.03E+02 | 1.51E+03 | 2.07 | 59.40 | 141.09 | 141.27 | 1.67E+02 | 4.16E+02 | 0.57 | 16.41 |
| 9 | 157.12 | 157.26 | 4.77E+02 | 1.00E+03 | 1.37 | 39.44 | 155.11 | 155.25 | 2.19E+02 | 4.59E+02 | 0.63 | 18.10 |
| 10 | 171.14 | 171.14 | 7.48E+02 | 1.27E+03 | 1.74 | 50.12 | 169.12 | 169.17 | 4.45E+02 | 7.57E+02 | 1.04 | 29.83 |
| 11 | 185.15 | 185.23 | 7.03E+02 | 1.06E+03 | 1.45 | 41.58 | 183.14 | 183.28 | 3.23E+02 | 4.85E+02 | 0.66 | 19.11 |
| 12 | 199.17 | 199.27 | 4.26E+02 | 5.54E+02 | 0.76 | 21.82 | 197.15 | 197.26 | 6.12E+02 | 7.96E+02 | 1.09 | 31.36 |
| 13 | 213.19 | 213.27 | 3.52E+02 | 4.22E+02 | 0.58 | 16.64 | 211.17 | 211.24 | 1.13E+03 | 1.36E+03 | 1.86 | 53.49 |
| 14 | 227.20 | 227.28 | 4.69E+02 | 5.16E+02 | 0.71 | 20.33 | 225.19 | 225.24 | 1.08E+03 | 1.19E+03 | 1.63 | 46.73 |
| 15 | 241.22 | 241.26 | 4.72E+02 | 4.72E+02 | 0.65 | 18.61 | 239.20 | 239.25 | 8.84E+02 | 8.84E+02 | 1.21 | 34.84 |
| 16 | 255.23 | 255.27 | 6.11E+02 | 6.11E+02 | 0.84 | 24.08 | 253.22 | 253.24 | 7.75E+02 | 7.75E+02 | 1.06 | 30.53 |
| 17 | 269.25 | 269.26 | 6.93E+02 | 6.93E+02 | 0.95 | 27.31 | 267.23 | 267.26 | 6.26E+02 | 6.26E+02 | 0.86 | 24.66 |
| 18 | 283.26 | 283.28 | 6.45E+02 | 6.45E+02 | 0.88 | 25.43 | 281.25 | 281.30 | 6.19E+02 | 6.19E+02 | 0.85 | 24.41 |
| 19 | 297.28 | 297.29 | 5.88E+02 | 5.88E+02 | 0.81 | 23.17 | 295.26 | 295.28 | 6.42E+02 | 6.42E+02 | 0.88 | 25.29 |
| 20 | 311.30 | 311.28 | 4.90E+02 | 4.90E+02 | 0.67 | 19.31 | 309.28 | 309.30 | 5.78E+02 | 5.78E+02 | 0.79 | 22.78 |
| 21 | 325.31 | 325.30 | 4.03E+02 | 4.03E+02 | 0.55 | 15.88 | 323.30 | 323.31 | 5.18E+02 | 5.18E+02 | 0.71 | 20.41 |
| 22 | 339.33 | 339.31 | 3.64E+02 | 3.64E+02 | 0.50 | 14.36 | 337.31 | 337.31 | 4.69E+02 | 4.69E+02 | 0.64 | 18.46 |
| 23 | 353.34 | 353.29 | 3.29E+02 | 3.29E+02 | 0.45 | 12.95 | 351.33 | 351.32 | 4.22E+02 | 4.22E+02 | 0.58 | 16.61 |
| 24 | 367.36 | 367.28 | 2.58E+02 | 2.58E+02 | 0.35 | 10.17 | 365.34 | 365.29 | 3.37E+02 | 3.37E+02 | 0.46 | 13.27 |
| 25 | 381.37 | 381.25 | 2.03E+02 | 2.03E+02 | 0.28 | 8.00 | 379.36 | 379.29 | 2.57E+02 | 2.57E+02 | 0.35 | 10.11 |
| 26 | 395.39 | 395.25 | 1.61E+02 | 1.61E+02 | 0.22 | 6.36 | 393.37 | 393.26 | 1.72E+02 | 1.72E+02 | 0.24 | 6.76 |
| 27 | 409.40 | 409.25 | 1.33E+02 | 1.33E+02 | 0.18 | 5.22 | 407.39 | 407.25 | 1.18E+02 | 1.18E+02 | 0.16 | 4.67 |
| 28 | 423.42 | 423.24 | 9.86E+01 | 9.86E+01 | 0.14 | 3.89 | 421.40 | 421.24 | 9.78E+01 | 9.78E+01 | 0.13 | 3.85 |
| 29 | 437.44 | 437.24 | 7.16E+01 | 7.16E+01 | 0.10 | 2.82 | 435.42 | 435.23 | 7.71E+01 | 7.71E+01 | 0.11 | 3.04 |
| 30 | 451.45 | | | | | | 449.44 | | | | | |

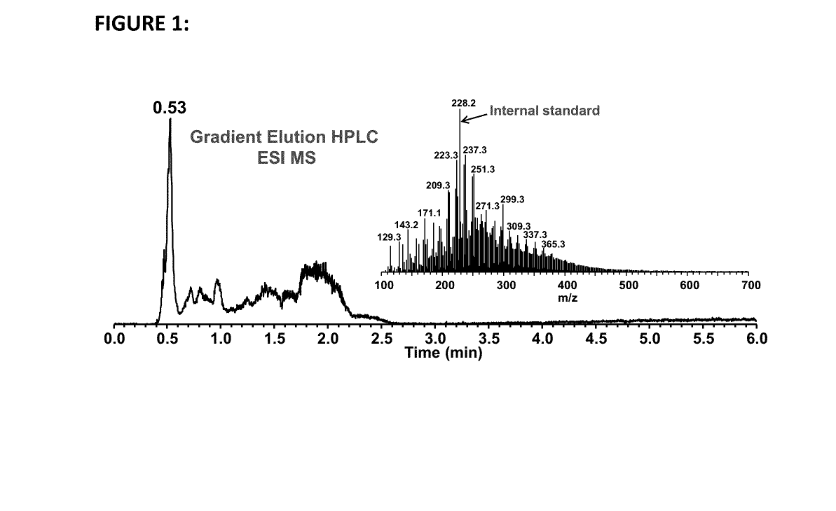

Several figures are provided to demonstrate the advantageous features of the inventive process. FIG. 1 shows a representative negative ESI total ion chromatogram trace of a sample produced by gradient elution HPLC, which shows several peaks that eluted from the HPLC column from about 0.4-2.6 minutes. The insert shows the associated mass spectra across the total ion chromatogram trace. The mass spectrum across the chromatogram shows numerous peaks corresponding to naphthenic acids, together with a peak corresponding to the spiked internal standard (m/z=228.2).

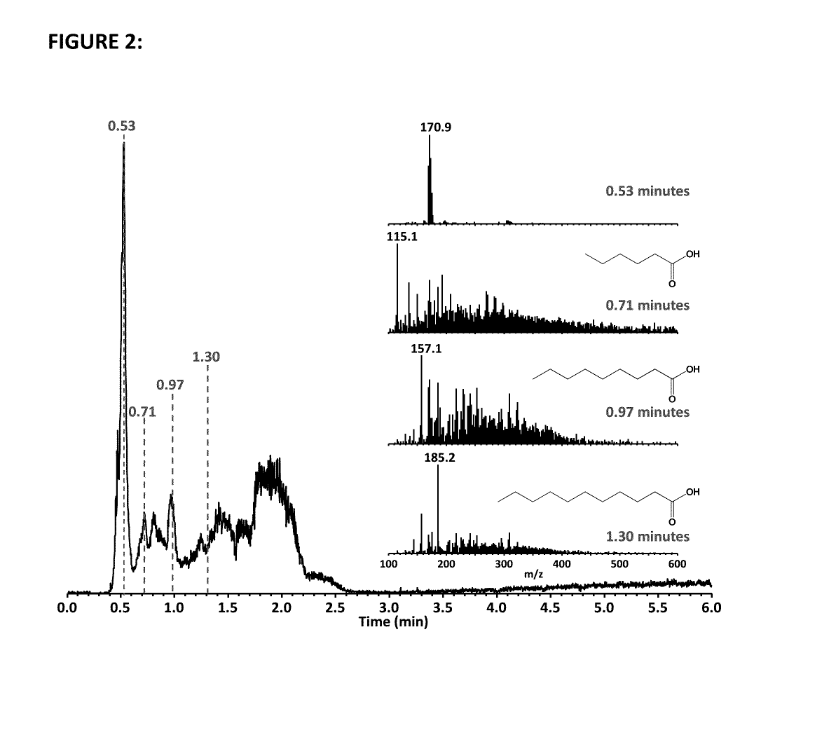

FIG. 2 demonstrates a more detailed analysis of the total ion chromatogram trace shown in FIG. 1. Mass spectra were obtained of chromatogram peaks eluting at 0.53, 0.71, 0.97, and 1.30 minutes, respectively. The spectrum of the first chromatogram peak shows an abundant early eluting species at 0.53 minutes. The second, third and fourth inserts in FIG. 2 shows mass spectra observed at various other retention periods: 0.71, 0.97 and 1.3 minutes. The most abundant ion signal in each spectrum (m/z=115.1, 157.1, and 185.2, respectively) corresponds to the acyclic NA of n=6, 9, and 11, as exemplified by the structure depicted immediately above each spectrum. Each of these spectra also show several other peaks corresponding to co-eluting NAs possessing similar retention characteristics, indicating that the NAs have similar ionization efficiency and are all ionized and detected (i.e., negligible signal suppression of each other).

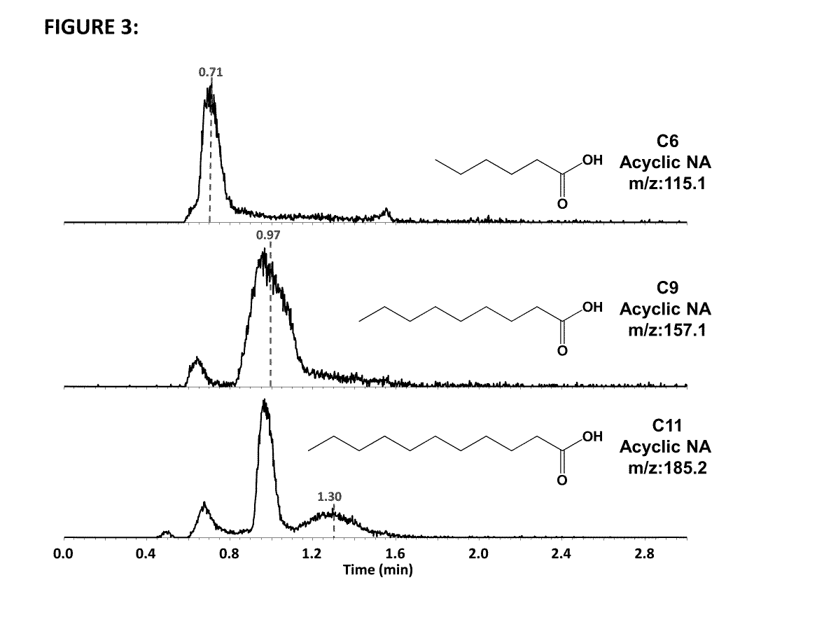

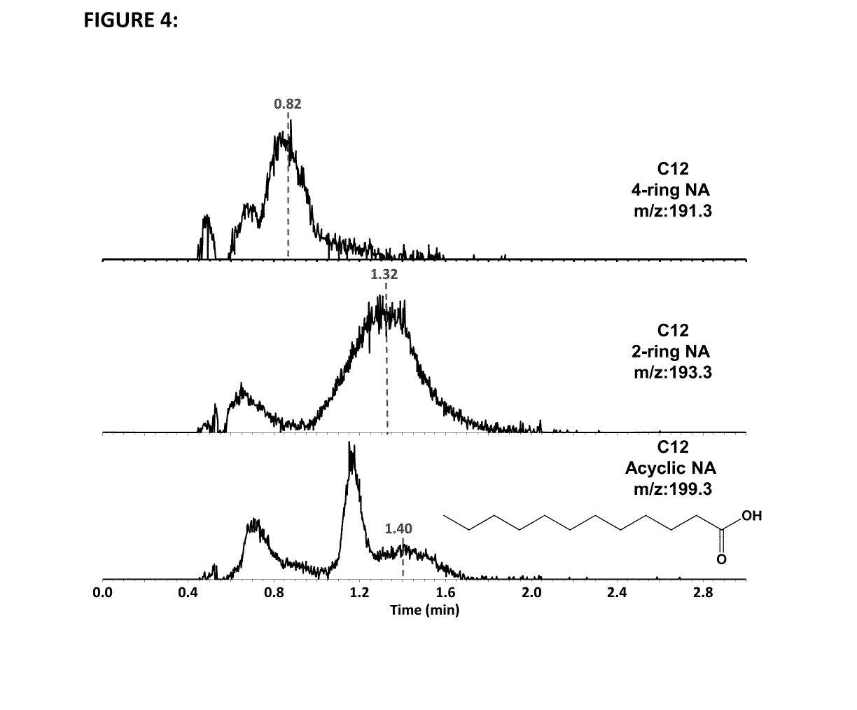

FIGS. 3 and 4 demonstrate the effectiveness of the processes detailed herein at effectively separating and quantitating various NA species from a refinery-produced water sample. FIG. 3 shows selected gradient elution HPLC ESI chromatogram traces corresponding to acyclic NAs possessing 6, 9 and 11 carbons, respectively. The retention periods for each of these straight chain NAs is highlighted in each chromatogram (0.71, 0.97, and 1.30 min., respectively), and a typical NA molecular structure for each is illustrated above each chromatogram. As one would expect, the results indicate an increase in the retention time with increasing chain length, while a progressive decrease in peak sharpness with increasing carbon number indicates the presence of an increased number of isomers.

FIG. 4 shows the selected ESI chromatogram traces for three detected NA species having the same carbon number (n=12) but different z numbers (−8, −4, and 0) corresponding to NAs structures with 4-rings, 2-rings and acyclic, respectively. The NA molecular structures become more compact with an increasing number of rings, which is evident by the decreased retention period on the HPLC column.

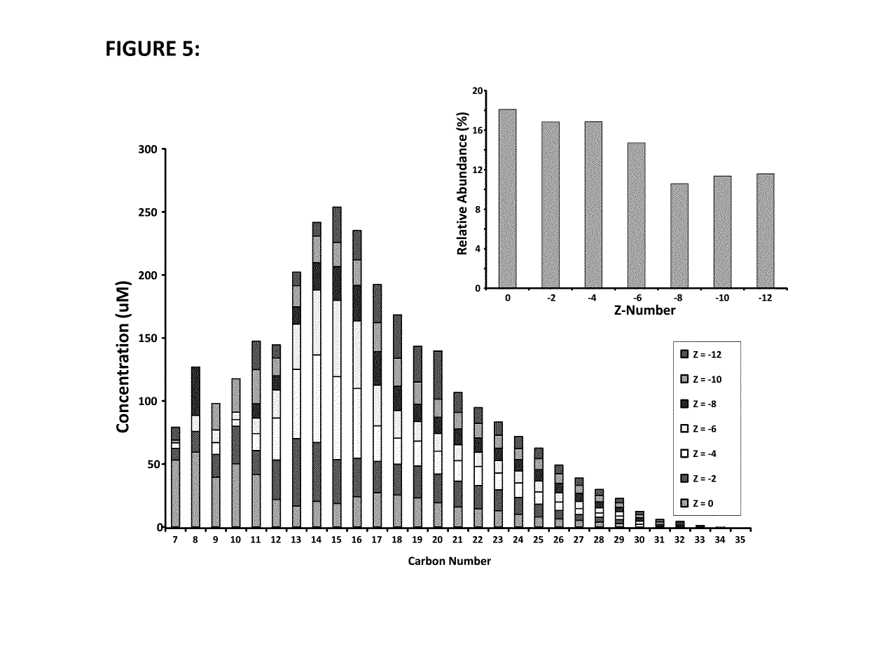

FIG. 5 is a stacked bar graph that depicts the distribution of NAs (by total carbon number) in a refinery-produced water sample. The bar height represents the total concentration of NA (μM) at each carbon number, while the various shadings depict the relative abundance of NAs having one or more ring structures (indicated by decreasing z number). The insert bar graph in FIG. 5 depicts the relative abundance of NAs that were acyclic (z=0), or had one or more ring structures (z=−2, −4, −6, etc.) irrespective of total carbon number.

The methods described in detail herein provide several advantages versus prior methods for quantitating employs on-line HPLC gradient elution separation of NAs from sample impurities, eliminating ionization suppression to generate abundant signal for all NAs present in each sample. The method requires no chemical modification of the sample (such as derivitization, for example) or extraction of the sample, thereby allowing rapid sample throughput and decreased cost to conduct the method. The HPLC reverse-phase gradient elution separation minimizes or eliminates matrix suppression effects to allow detection of all NAs present in various samples with superior detection thresholds that are as much as 350-fold lower than conventional methods and increased specificity compared to conventional direct injection. In the present inventive processes, naphthenic acid identification is based not only on HPLC separation (which minimizes co-elution of NA species), but also on measurement of mass-to-charge ratio resulting in minimal ambiguity in NA identification even for co-eluting NAs.

In addition to improved sensitivity and specificity, the inventive methods allows accurate quantitation of NA levels by incorporating a unique response correction for NA signal magnitudes based on prior analysis of a mixture of straight-chain C8-C24 naphthenic acids. This response correction is particularly important for correcting the mass spectrometry signal of NAs containing seven or less carbons to provide accurate quantitation. An additional advantage is that the HPLC gradient elution separation allows resolution of isomeric NAs to provide information on the relative abundance of each isomer. Such information can be useful in situations where one NA isomer might prove more corrosive to process equipment than the other isomer.

The following examples are intended to be illustrative of specific embodiments in order to teach one of ordinary skill in the art how to make and use the invention.

### Example 1

An HPLC system was utilized that incorporated an Accela™ pump connected to a C18 liquid chromatography (LC) column (Zorbax SB-C18 2.1×50 mm, 3.5 μm, Agilent Technologies, Santa Clara Calif.). A process water sample or crude oil extract was spiked with an isotopically labeled internal standard and injected onto the LC column, with gradient elution of analytes in the reverse phase mode. Sample impurities were separated from naphthenic acid (NA) species and individual NAs separated from each other on the column based on their carbon number, number of naphthenic rings, and extent of alkyl branching. HPLC separation of naphthenic acids from other sample components eliminated the ionization suppression effect allowing detection of all naphthenic acid species in the sample, and allowing clear chromatographic resolution of NA isomers.

The LC mobile phase comprised Optima LCMS HPLC grade water and methanol. Isotope-labeled myristic acid-1-¹³C was added as an internal standard.

Effluent from the LC column was nebulized by electrospray into highly-charge droplets, then ionized an electrospray ionization source to highly charged droplets from which negative ions then evaporated. The ions were then directed into the inlet of a linear quadrupole ion trap mass spectrometer (LTQ XL, Thermo Scientific, Sans Jose, Calif.) followed by detection of ions to generate a spectrum generated based on mass-to-charge ratio of the ions.

TABLE 1
LC gradient and mass spectrometer
settings used for all experiments.

| LC Gradient | | | MS Settings | | |
|---|---|---|---|---|---|
| Time | % A | % B | | | |
| (min.) | (H₂O) | (MeOH) | Variable | Set value | |
| 0.0 | 30 | 70 | ESI voltage | 5 | kV |
| 0.2 | 20 | 80 | Capillary | 250° | C. |
| | | | temperature | | |
| 1.0 | 18 | 82 | Capillary voltage | −25 | V |
| 4.0 | 16 | 84 | Tube lens voltage | −25 | V |

| | | | | |
|---|---|---|---|---|
| 18.0 | 0 | 100 | m/z range | 100-700 |
| 25.0 | 0 | 100 | Ion trap target | 100,000 |

Accurate quantitation of the NAs in the sample was achieved in two steps by 1) normalizing values according to the known quantity of isotope-labeled internal standard added to the sample, and 2) correcting the m/z signal magnitude utilizing a response factor empirically determined by prior separation and mass spectral analysis of a C6-C24 mixture of NAs in the same system to establish the relationship between m/z signal magnitude versus n number. This correction has been described in greater detail above.

In closing, it should be noted that the discussion of any reference is not an admission that it is prior art to the present disclosure, in particular, any reference that may have a publication date after the priority date of this application. At the same time, each and every claim below is hereby incorporated into this detailed description or specification as a additional embodiments of the present invention.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims while the description, abstract and drawings are not intended to limit the scope of the invention. The invention is specifically intended to be as broad as the claims below and their equivalents.

We claim:

1. A process for quantitating one or more naphthenic acids in a liquid sample, comprising:
a. Subjecting a portion of the liquid sample to reverse phase high performance liquid chromatography utilizing a chromatography column capable of separating molecules having a molecular weight of 2,000 or less to produce a chromatography effluent comprising at least partially separated naphthenic acids, each naphthenic comprising from 2 to 25 carbons;
b. Directing the chromatography effluent to an electrospray ionization source to produce negatively charged naphthenic acid ions that are analyzed on a mass spectrometer to produce a raw mass spectrum;
c. Correcting the raw mass spectrum by multiplying the intensity of one or more spectral peaks corresponding to the one or more naphthenic acids by a response factor,
wherein the response factor is empirically-determined via measurement of the relative mass spectrum peak intensity of known quantities of carboxylic acid standards comprising between 2 and 25 carbons, the response factor being dependent upon the number of carbons in the carboxylic acid standards, the number of ring structures in the carboxylic acid standards, or both,
wherein the response factor corrects the signal magnitude of the one or more spectral peaks corresponding to the one or more naphthenic acids, thereby allowing more accurate quantitation of the one or more naphthenic acids in the liquid sample.

2. The process of claim 1, additionally comprising correcting the raw mass spectrum by subtracting a blank mass spectrum, or the average of two or more blank mass spectrums, thereby allowing more accurate quantitation of the one or more naphthenic acids.

3. The process of claim 1, wherein chromatography effluent is eluted from the column utilizing an increasing organic solvent gradient.

4. The process of claim 3, wherein the organic solvent is methanol.
